(19) United States
(12) Patent Application Publication
(10) Pub. No.: US 2021/0052300 A1
(43) Pub. Date: Feb. 25, 2021

(54) CANNULA AND OBTURATOR SYSTEM

(71) Applicant: Rebound Therapeutics Corporation, Irvine, CA (US)

(72) Inventor: Donald Joseph Fuller, Irvine, CA (US)

(73) Assignee: Rebound Therapeutics Corporation, Irvine, CA (US)

(21) Appl. No.: 16/550,163

(22) Filed: Aug. 23, 2019

(51) Int. Cl. A61B 17/34; A61B 1/05; A61B 90/35

(52) U.S. Cl. CPC A61B 17/3423; A61B 1/05; A61B 90/361; A61B 90/35; A61B 17/3415; A61B 17/3494

(57) ABSTRACT

A cannula system and method for accessing a blood mass in the brain. The system comprises a cannula with a camera mounted on the proximal end of the cannula with a view into the cannula lumen and the surgical field below the lumen, an obturator with a small diameter shaft and a large diameter tip which is optically transmissive. The obturator tip includes a ring seal to prevent blood from entering the cannula lumen during insertion into a bloody surgical site.

### FIELD OF THE INVENTIONS

The inventions described below relate to the field of minimally invasive brain surgery.

### BACKGROUND OF THE INVENTIONS

Stroke is a common cause of death and disabling neurologic disorder. Approximately 700,000 patients suffer from stroke in the United States every year. Hemorrhagic stroke accounts for 20% of the annual stroke population. Hemorrhagic stroke is due to a rupture of a blood vessel in the brain, causing bleeding into the brain tissue and resulting in a hematoma (a blood mass) in the brain. Prompt removal of the blood mass is necessary to limit or prevent long-term brain injury.

Clear visualization and imaging of the blood mass and any surrounding surgical field facilitates removal of the blood mass. Removal and visualization can often be accomplished through a cannula and obturator assembly, placed through a hole drilled in the skull near the site of the hematoma. The site of the hematoma can be accurately identified using a CT scan.

In our prior U.S. Pat. No. 10,376,281, we disclose a cannula system with a cannula, a proximally mounted camera, and an obturator with a narrow shaft and large obturator tip viewable with the camera from the proximal end of the cannula. Occasionally, blood may enter the cannula lumen during insertion into the body to access a surgical workspace.

### SUMMARY

The devices and methods described below provide for improved visualization of the brain during minimally invasive surgery. In a cannula system with a cannula, a proximally mounted camera, and an obturator with a narrow shaft and large obturator tip viewable with the camera from the proximal end of the cannula, the obturator tip is fitted with a circumferential seal for preventing blood from a surgical workspace from leaking through any small clearance between the obturator tip and the cannula tube and obscuring the proximal surface of the obturator tip.

### BRIEF DESCRIPTION OF THE DRAWINGS

FIG. 1 illustrates a patient with a blood mass in the brain that necessitates surgical intervention, with a cannula which has been inserted into the brain, with the distal end of the cannula proximate the blood mass and an obturator tip extending into the blood mass.

FIG. 2 illustrates a cannula, camera and obturator system.

### DETAILED DESCRIPTION OF THE INVENTIONS

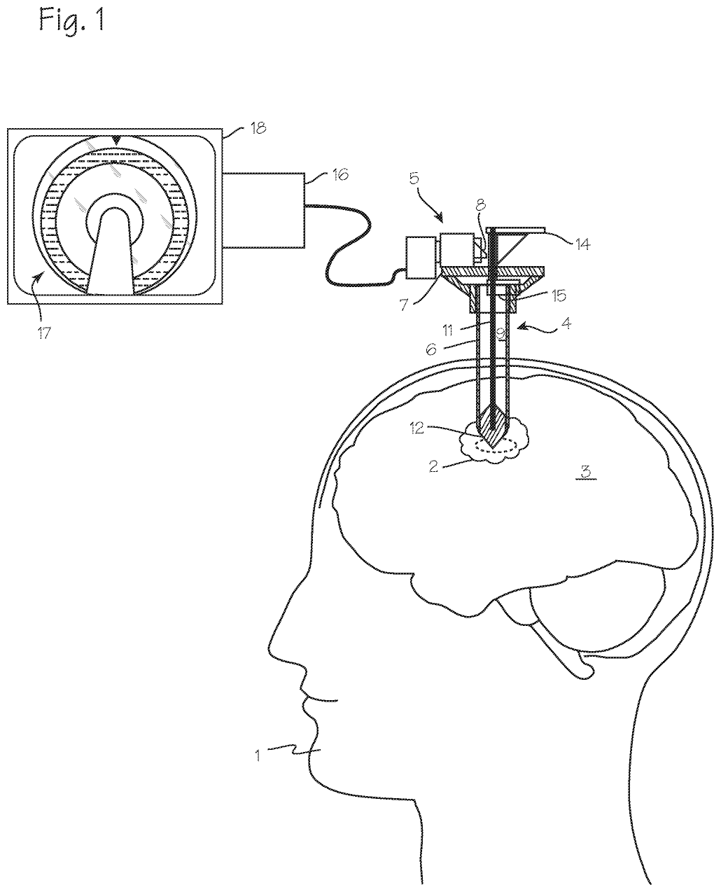

FIG. 1 illustrates a patient 1 with a blood mass 2 in the brain 3 that necessitates surgical intervention. A cannula 4 has been inserted into the brain, with the distal end of the cannula proximate the blood mass. A camera 5 is mounted on the proximal rim of the cannula, with a portion of the camera overhanging the rim of the cannula and disposed over the lumen of the cannula, and is operable to obtain video or still images of the blood mass or other tissue at the distal end of the cannula.

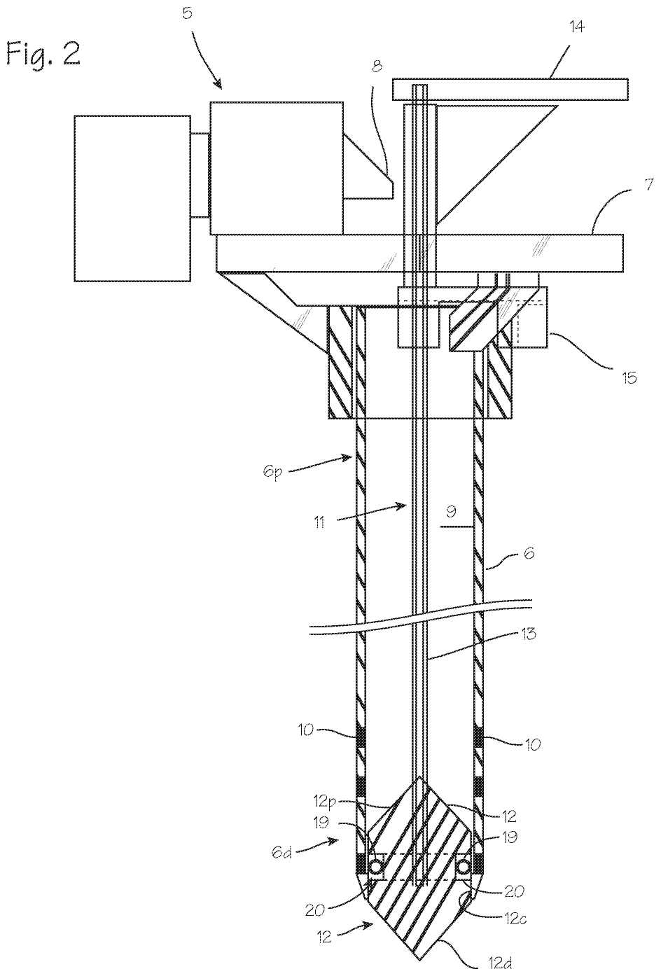

The cannula comprises a cannula tube 6, with a distal end 6d adapted for insertion into the body of the patient, and the proximal end 6p which remains outside the body during use. A camera 5 is mounted on the proximal end 6p of the cannula tube. A mounting structure 7 is secured to the proximal end of the cannula. The camera may include or be fitted with a prism 8, a reflector or other mirror structure or optical element, overhanging the lumen 9 of the cannula tube. If the camera is small compared to the cannula lumen, the camera may be used without the prism or reflector, and may be oriented with its viewing axis aligned along the long axis of the cannula. The light necessary to provide good visualization of the blood mass, and obtain images of the blood mass, may be provided by light sources 10 (LED's or other light source) disposed at the distal end 6d of the cannula tube, at or proximate the distal opening (See FIG. 2). The light sources may instead be disposed at the proximal end of the cannula tube and the light may be transmitted through the open lumen of the cannula, or may be transmitted through optical fibers, or, if the cannula is made of a transparent material, the light may be transmitted down the walls of the cannula tube to exit the distal end of the cannula wall to illuminate the blood mass.

FIG. 2 also illustrates the obturator 11. The obturator comprises the obturator tip 12, shaft 13, handle 14, and mounting structure 15. The obturator tip is a solid structure with a conically convex distal surface 12d, a conically convex proximal surface 12p, and an axially short circumferential surface 12c. The tip, in the region of the circumferential surface, has an outer diameter (a transverse diameter, along a plane perpendicular to the long axis of the cannula, and corresponding to the transverse cross sectional diameter of the cannula) that closely matches the inner diameter of the cannula, but allows easy longitudinal translation of the tip through the lumen of the cannula.

The small cross-section obturator shaft 13 is much smaller than the inner diameter of the cannula, and much smaller that the diameter of obturator tip, affording a sizable annular or circular space between the shaft and the cannula wall to provide good visibility (from the camera) of the proximal surface of the obturator tip. Thus, with the obturator positioned with the cannula tube, the proximal surface of the obturator tip is visible to the camera (or another imaging device) from the proximal end of the cannula tube through an annular space between the shaft and the cannula tube.

Lights, if necessary, may be provided in the cannula to illuminate the distal end of the obturator tip and cannula or tissue near the distal end of the cannula (lighting may instead be provided from a source outside the assembly, or from lights mounted on the proximal end of the cannula or any combination of the foregoing). Light reflected by tissue near the distal surface of the obturator tip passes through the obturator and out of the proximal surface of the obturator tip, so that a surgeon inserting or manipulating the assembly can easily see that the obturator tip is near brain tissue (which is white to gray) or blood (which is red to black).

The obturator tip is optically transmissive, not optically opaque, and may be optically transparent or optically translucent. The transmittance of the tip need only be adequate, in the visible spectrum, to pass the color of tissue in contact with the distal surface, given the brightness of any illumination provided by the light sources, to provide enough transmitted light to the camera and/or eye of the surgeon to allow the color of tissue around the tip to be discerned from light transmitted through the proximal surface of the tip.

The proximal surface of the tip, which tapers to a small diameter in the proximal direction, also provides for clearance of the tip when the obturator must be removed to make room for other devices.

In use, a surgeon inserts the cannula 4 with an obturator 11 into the patient's brain until the distal end 6d of the cannula is sufficiently close to tissue 2 for surgery. While inserting the cannula and obturator, the surgeon operates the camera and control system 16 to display an image 17 of the cannula lumen and structures at the distal end of the cannula on a display screen 18. Image data from camera 5 is transmitted to the display to provide an image or images of the structures at the distal end of the cannula through lumen 9 and the proximal surface of the obturator tip. The display may be operated by a control system 16 which is operable to receive image data from the camera, transmit the image data to the display, and also add additional images to the display such as markers, cursors, and indicia of patient data. If the cannula lumen is large, the surgeon may directly view the proximal surface of the obturator tip to view the brain or blood proximate the distal surface of the obturator tip.

The cannula and obturator are intended for use while observing tissue distal to the obturator tip from the proximal end of cannula, using the imaging system. A small clearance between the obturator tip and the inner wall of the cannula may allow blood to pass into the cannula and cover the proximal surface of the obturator tip, so that a user cannot make use of this feature. To address this problem, the obturator shown in FIGS. 1 and 2 includes a seal 19 surrounding the obturator tip. The seal is disposed about a circumferential surface of the obturator tip. Preferably, the seal is partially disposed within a circumferential groove 20, and the seal is set in groove. The seal prevents blood from the body, if present in the surgical site, from leaking into the cannula lumen and thus obscuring the camera view of the obturator tip, or viewing the proximal end of the obturator tip from a viewpoint at the proximal end of the cannula, through the lumen of the cannula tube.

The seal may comprise an O-ring, an elastomeric band, or gasket comprising elastomers, fluoropolymers (PTFE, for example) or rubbers.

While the preferred embodiments of the devices and methods have been described in reference to the environment in which they were developed, they are merely illustrative of the principles of the inventions. The devices may be used for various intracerebral procedures such as intra-ventricular hemorrhage procedures, neuro-stimulation procedures, and tumor resection. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A cannula system for accessing a blood mass in the brain of a patient, said cannula system comprising:
a cannula comprising a cannula tube with a proximal end and a distal end and a lumen extending from the proximal end to the distal end;
an obturator comprising an obturator shaft having a proximal end and a distal end, and an obturator tip disposed on the distal end of said obturator shaft, said obturator tip being optically transmissive, and having a proximal surface and a tapered distal surface, said obturator having a transverse diameter closely matching the lumen of the cannula tube, said obturator being slidable within the cannula tube, and positionable within the cannula tube such that the proximal end of the obturator shaft extends proximally out of the cannula tube proximal end while the tapered distal surface extends out of the cannula tube distal end, wherein the obturator shaft has a transverse cross section smaller than a transverse diameter of the obturator tip, whereby the proximal surface of the obturator tip is visible from the proximal end of the cannula tube, through the lumen of the cannula tube, when the obturator tip is disposed within the cannula tube such that the tapered distal surface extends out of the cannula tube distal end; and
a seal disposed about a circumferential surface of the obturator tip.

2. The cannula system of claim 1, further wherein:
the obturator tip has a groove in the circumferential surface, and the seal is set in the groove.

3. The cannula system of claim 1, further comprising:
a camera assembly secured to the proximal end of the cannula, with a portion of the camera assembly overhanging and partially obstructing the lumen, said camera configured to obtain images of the proximal surface of the obturator tip.

4. The cannula system of claim 3, further comprising:
a display operable to display images from the camera assembly, where said images from the camera assembly include an image of an inner wall of the cannula tube proximate the proximal surface of the obturator tip, an image of the proximal surface of the obturator tip, and an image of the obturator shaft; and
a control system operable to receive image data from the camera assembly and transmit corresponding image data to the display, and transmit an image of a marker to the display, said marker corresponding to an index on the cannula or obturator.

5. The cannula system of claim 3, wherein:
the camera assembly has a viewing axis, and the distal-most optical surface of the camera assembly is disposed over the proximal end of the cannula tube and angled to aim the viewing axis toward the proximal surface of the obturator tip.

6. The cannula system of claim 3, wherein:
the obturator tip is configured to pass into the cannula lumen while the portion of the camera assembly overhanging the lumen is in place overhanging the lumen.

7. The cannula system of claim 1, wherein:
with the obturator positioned with the cannula tube, the proximal surface of the obturator tip is visible from the proximal end of the cannula tube through an annular space between the shaft and the cannula tube.

8. The cannula system of claim 3, wherein:
with the obturator positioned with the cannula tube, the proximal surface of the obturator tip is visible, to the camera assembly, from the proximal end of the cannula tube through an annular space between the shaft and the cannula tube.
